(19) United States
(12) Patent Application Publication
(10) Pub. No.: US 2010/0267102 A1
(43) Pub. Date: Oct. 21, 2010

(54) WASTE DISTRIBUTION, CONVERSION, AND UTILIZATION

(75) Inventors: Ryan Begin, Waltham, MA (US); Shane Eten, Boston, MA (US)

Correspondence Address: LANDO & ANASTASI, LLP, ONE MAIN STREET, SUITE 1100, CAMBRIDGE, MA, 02142, US

(73) Assignee: Feed Resource Recovery, Inc., Boston, MA (US)

(21) Appl. No.: 12/424,785

(22) Filed: Apr. 16, 2009

(51) Int. Cl. C12P 5/00; C12M 1/00; C02F 11/04

(52) U.S. Cl. 435/166; 435/290.1; 435/290.4

(57) ABSTRACT

Food wastes streams may be managed efficiently by co-locating a waste-processing facility including a pulper and an anaerobic bioreactor with a food distribution facility.

### TECHNICAL FIELD

In various embodiments, the invention relates to waste recovery and conversion, and more particularly to improved methods and systems for recovering biodegradable waste for conversion into useful products.

### BACKGROUND

In nature, plants extract nutrients from the soil to fuel their growth and, if not consumed, decompose into simpler forms of matter (e.g., CO₂ and soil nutrients), which become energy for the next generation of growing plants. As such, natural organic processes provide a “closed-loop” system of energy and nutrient cycles.

In contrast, in the food industry, waste is abundant. Over 40% of the food produced in the U.S. goes to waste, and traditional waste-disposal practices bury 97% of this waste in landfills, which in turn release methane (which is 25 times more harmful than CO₂). This “cradle-to-grave” system has over-taxed local landfills and has resulted in a growing movement to ban food waste from landfills. In addition, increased disposal site distances, combined with rising oil prices, make transporting waste a costly proposition (e.g., approximately $100-150/ton). This system of waste disposal has also encouraged farmers to purchase increasingly expensive and environmentally harmful chemicals in order to replace lost soil nutrients due to industrial farming practices. The result is an “open-loop” system that does not properly value or recover waste.

### SUMMARY OF THE INVENTION

In various embodiments, the present invention relates to systems and methods that use waste-conversion technology to turn previously discarded food waste and other biodegradable waste into fertilizer and/or energy. More particularly, waste-conversion methods can be used to generate energy usable by the waste generator (e.g., supermarkets and restaurants) and/or provide a source of affordable fertilizer for local farmers. As a result, economic and environmental value may be recovered from previously discarded waste efficiently and economically.

In one aspect, the invention relates to a system, including a pulper and an anaerobic membrane bioreactor, for processing solid and/or liquid waste that contains biodegradable material. Biodegradable material can be broken down by living organisms. Typically, it contains largely material from plant or animal sources, but it may also include non-biological materials such as, e.g. biodegradable packaging materials. In some embodiments, the system is used for processing food waste, which may include both biodegradable components (e.g., meat, produce) and non-biodegradable components (e.g., bones, scales, packaging materials). Non-biodegradable material, as the term is used herein, includes material which biodegrades only slowly, i.e., on time-scales which render its processing in a bioreactor unfeasible.

The pulper removes non-biodegradable material from the waste stream, and pulps the biodegradable material. In the anaerobic membrane bioreactor, microorganisms then produce biogas and anaerobic effluent from the pulped biodegradable material. In various embodiments, the anaerobic effluent includes a sludge and a permeate containing nutrients and/or minerals. The anaerobic bioreactor includes a membrane for retaining the microorganisms and a solid portion of the biodegradable material in the bioreactor. The membrane may be submerged in the main bioreactor container. Alternatively, the material contained in the main container may be cycled through a separate membrane unit. The pulper, by removing non-biodegradable material from the waste stream before it enters the anaerobic membrane bioreactor, protects the membrane.

In some embodiments, the system further includes a hydrocyclone that couples the pulper to the anaerobic membrane bioreactor. The hydrocyclone removes grit from a suspension of the pulped biodegradable material prior to its introduction into the bioreactor, thereby further protecting the membrane. The system may also include a heater for heating an interior of the pulper. Heating the pulper may serve to solubilize the biodegradable material. In some embodiments, the system further includes waste containers, for transporting the waste to the pulper, which contain microorganisms that partially degrade the waste during transport.

In another aspect, the invention relates to a method for processing waste by removing non-biodegradable material from the waste and pulping the biodegradable material; and anaerobically processing the pulped biodegradable material. The anaerobic processing step involves exposing the pulped biodegradable material to microorganisms for producing biogas and anaerobic effluent therefrom, and retaining the microorganisms. The step may take place in an anaerobic membrane bioreactor, and the removal of non-biodegradable material may serve to protect the membrane of the anaerobic membrane bioreactor.

In yet another aspect, the invention relates to a method of managing a waste stream including biodegradable material. The method involves providing a waste-processing facility co-located with a distribution facility. The distribution facility receives food products from one or more producers (e.g., local farmers), and distributes the food products to at least one retailer (e.g., a grocery store). The waste-processing facility includes a pulper for pulping biodegradable waste, and an anaerobic bioreactor for producing biogas and anaerobic effluent from the pulped biodegradable waste. The bioreactor may be an anaerobic membrane bioreactor. In certain embodiments, the waste-processing facility is monitored from a remote location.

In some embodiments, fertilizer and/or dilution water for the pulper are generated from the anaerobic effluent. The biogas may be converted into fuel and/or electrical energy. Energy generated from the biogas may also be used to heat the pulper and/or the anaerobic bioreactor.

The method further involves using the same vehicles to deliver food products from the distribution facility to the retailer and to return biodegradable waste generated by the retailer to the waste-processing facility. The waste may be transported from the retailer to the waste-processing facility in waste containers that are bioaugmented; in this way, the waste is partially degraded during transport. In some embodiments, the method also includes using the same vehicles to transport the food products from the producer to the distribution facility and to deliver fertilizer generated from the anaerobic effluent to the producer.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic view of an exemplary system for distributing food products and collecting and processing biodegradable waste in accordance with one embodiment of the invention;

FIG. 2A is a schematic view of an anaerobic digestion process in accordance with one embodiment of the invention;

FIG. 2B is a table of digestate attributes for materials processed by a biodegradable-waste-processing facility, in accordance with one embodiment of the invention;

FIG. 3 is a schematic view of a waste-processing facility in accordance with one embodiment of the invention;

FIG. 4 is a schematic view of a pulper and contaminant removal system in accordance with one embodiment of the invention;

FIGS. 5A and 5B are schematic views of anaerobic membrane bioreactors in accordance with various embodiments of the invention; and

FIG. 6 is a schematic view of a remote telemetry system for a waste-processing facility in accordance with one embodiment of the invention.

### DESCRIPTION

In general, the present invention relates to the recycling of biodegradable waste to create fertilizer and/or energy. It allows biodegradable waste generators to turn what was once a liability (e.g., food waste to be discarded) into valuable resources. Various embodiments utilize existing transport infrastructure to transport the waste, thus facilitating recycling at little or no additional cost or environmental impact. By integrating waste-conversion technology with existing transport infrastructure, distributed energy generation units, and/or automation and remote management capabilities, certain embodiments provide an all-in-one waste management solution.

Biodegradable waste generators that may benefit from the systems and methods described herein include food retailers, such as supermarkets, food processors, restaurants, and canteens. Slim profit margins, high waste fees, and limited storage space, as well as image consciousness may drive these waste generators to seek clean technology solutions for waste management. Various embodiments of the invention allow them not only to minimize the amount of waste to be disposed of, but also to benefit from waste conversion into fertilizer and renewable energy. In many cases, food-waste generators can easily separate biodegradable from non-biodegradable waste, thus aiding in more efficient waste-processing downstream. However, this capability is not required, as various embodiments of the invention enable the processing of heterogeneous waste streams that include both biodegradable and non-biodegradable materials.

Various embodiments of the invention may be utilized to dispose of residual fats, oils, and grease (FOG), which are by-products that many food service, sales, and generator establishments need to manage. FOG occurs naturally in many foods such as meat. Oil and grease are also incorporated as ingredients into many recipes for bread, salads, and desserts, and are used as a medium for frying food. Thus, FOG is generated as a consequence of cooking. The increased development of central business districts encircled by suburban areas, the increasing mobility of our society, and decreased cooking at home have led to significant growth in the commercial food sector, including an increase of commercial areas containing high densities of restaurants and mall food courts, and an increase in ready-to-eat meals sold in supermarkets. As a result, FOG production through, e.g., ware washing, floor cleaning, and equipment sanitation, has increased likewise.

Sanitary sewer systems are neither designed nor equipped to handle the FOG that accumulates on the interior of the municipal sewer collection system pipes. To prevent FOG from reaching the sanitary sewer, a grease trap or grease separation device may be utilized. A grease separation device is a chamber or underground tank designed to let wastewater pass through, but to retain free or emulsified oil. FOG forms a free-floating layer on the water which can easily be removed by liquid waste haulers. The material that is collected is very difficult and expensive to dispose. Thus, a central location where supermarkets or their partners can dispose of their FOG provides a competitive advantage. FOG may be blended into a heterogeneous biodegradable waste stream so as to make up a portion of the chemical oxygen demand (COD) of the waste, and then fed into an anaerobic digester. The synergy created by a central anaerobic digestion facility that handles both food waste and FOG serves to consolidate the biodegradable wastes. Moreover, FOG has a significant potential for biogas production.

Food retailers are often supplied from a central distribution facility that receives the food products from a producer or wholesaler. Food products, as the term is used herein, includes, but is not limited to, fresh produce such as fruit and vegetables, grains, dairy products, meats, shelf-stable or containerized foods (e.g., canned soups), as well as animal feed. In general, the distribution facility collects the food products from the producer, such as a farmer, whole-sale distributor, or food factory, and ships the required quantities of food products to the retailer by truck, freight train, or other appropriate shipping means.

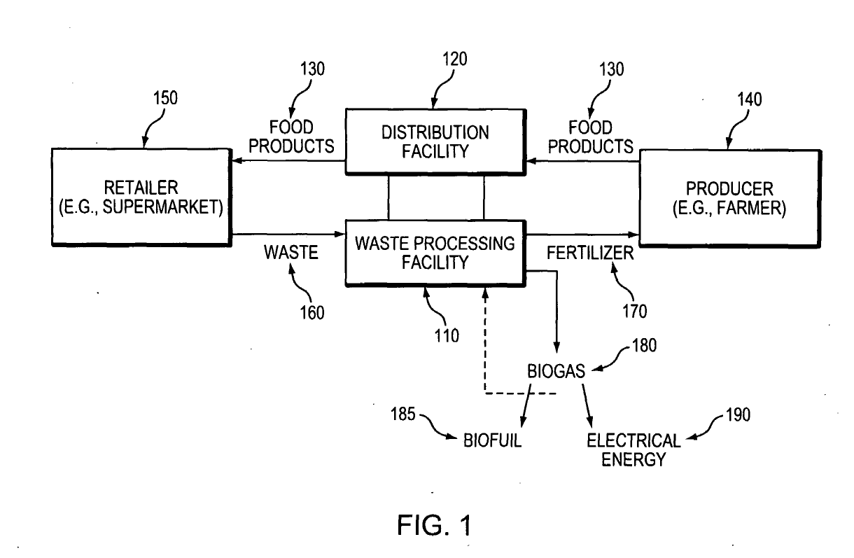

FIG. 1 illustrates an exemplary system 100 that integrates the delivery of food products to a retailer with the removal of waste from the retailer. In this embodiment, a waste-processing facility 110, described in more detail below, is located at or near (i.e., “co-located” with) a food product distribution facility 120. In operation, food products 130 are transported from a producer 140 to the distribution facility 120 for distribution to one or more retailers 150.

In one embodiment, the food products 130 are transported to a retailer 150 by truck. Once the food products have been delivered, the truck removes biodegradable waste 160 stored by the retailer (including, e.g., spoiled and unsold produce, biodegradable packaging material, and other associated waste), and hauls it to the waste-processing facility 110 on its return trip. As a result, the waste 160 is transported to the waste-processing facility 110 with substantially no additional environmental impact, as this occurs on the return leg of an existing delivery run.

In some embodiments, biodegradable waste 160 is accumulated by a retailer 150 in portable storage bins delivered to the retailer 150 by a truck during one food product delivery. When these are full, they are collected by the truck for return to the waste-processing facility 110 during a subsequent food product delivery. As a result, the truck delivers empty storage bins as part of a regularly scheduled delivery and collects fully loaded waste-storage bins for return to the waste-processing facility on the return trip. The storage bins may therefore be repeatedly reused as part of the delivery and recycling process. The storage bins may be manufactured from any appropriate material including, but not limited to, plastics, metals, or other appropriate material.

In certain embodiments, the waste-storage bins are bioaugmented to facilitate pretreatment of the waste in the containers during transport. For example, the containers may contain enzymes, enzyme-discreting fungus (such as trichoderma reesei), or other microorganism, that begin degrading the organic material. If different types of waste are stored separately, type-specific pretreatment may be performed. Meat waste may be treated, e.g., with lipase enzyme, and produce waste with white-rot fungus or trichoderma reesei.

Once the waste has been returned to the waste-processing facility 110, the biodegradable fraction can be processed into fertilizer 170 and/or biogas 180. By co-locating the waste-processing facility 110 with the distribution facility 120, waste 160 from multiple retailers 150 can be processed centrally by a single processing facility 110. Furthermore, because the waste 160 is transported to the distribution facility 120 on the return leg of scheduled deliveries to the retailers, this occurs without incurring additional transport costs.

In one embodiment, the waste-processing facility 110 includes a single, stand-alone waste-processing unit, which may be approximately 30′×8′×8′ in size. In an alternative embodiment, a number of coupled or separate waste-processing units may be utilized, depending upon the volume of waste being processed and the requirements of the system. Each waste-processing unit may be an automatic system that produces fertilizer, fuel, and/or energy upon insertion of organic waste, with few or no byproducts. Alternatively, different processing units may be linked in series or in parallel to produce the required quantities of fertilizer 170, fuel 185, and electrical energy 190 for the particular facility.

The fertilizers 170 derived from the biodegradable waste 160 may contain elements, such as micronutrients, that improve soil health and structure and that are not found in synthetic fertilizers. In some embodiments, the fertilizer 170 generated by the waste-processing unit 110 is distributed to the producers 140 (e.g., local farmers) in the same trucks that are used to pick up the food products 130 from the producer 140 and deliver it to the distribution facility 120. (These trucks may or may not be the same ones that travel to and from retailers.) This allows further exploitation of existing transport infrastructure in delivering the fertilizer 170 to the producer 140 without incurring additional transport costs, and therefore without additional environmental impact. This fertilizer 170 may then be used by the farmer to fertilize crops for subsequent distribution and sale.

In some embodiments, the biogas 180 generated in the waste-processing facility is further converted to biofuel 185 (in gas, liquid, or solid form) and/or electrical energy 190. The electrical energy 190 may be used directly to power the waste-processing facility 110, and/or may be distributed through the electrical grid. Heat or other forms of utilizable energy may also be generated by the processing facility 110 in addition to, or in place of, electrical energy.

In an alternative embodiment, a stand-alone waste-processing system is installed at the site of a waste generator (e.g., a produce retailer such as a supermarket). The system may be utilized with little behavior modification from current waste-handling practices while allowing the retailer to process the waste directly. In this embodiment, any electrical energy or fuel generated by the processing facility may be utilized directly by the retailer, or distributed through the electrical grid. Any fertilizer produced by the waste-processing facility can be delivered to a producer of produce through existing transport infrastructure, for example, by shipping the fertilizer to a produce distribution facility in trucks previously used to deliver produce to the retailer, and thereafter in (the same or different) trucks used to pick up produce from the producer and transport it to the distribution facility. In an alternative embodiment, the retailer can sell the fertilizer directly at its site.

Use of the systems and methods described herein may create revenue for users in the form of avoided costs. The primary avoided costs are electricity, waste management, depreciation, transportation fuel, and heat. Benefits may also be derived through government-based subsidies such as Renewable Energy Credits (RECs) and Carbon Credits. Favorable public-relations opportunities may also be gained. In addition, revenue may be created directly through the sale of the fertilizer, electrical energy, and/or fuel produced by the waste-processing facility.

The waste-management system described above requires a facility for processing biodegradable waste. Therefore, in one implementation, the waste-processing facility includes an anaerobic digester. Anaerobic digestion is the breakdown of organic material by microorganisms in the absence of oxygen. Although this process occurs naturally in landfills, anaerobic digestion usually refers to an artificially accelerated operation that processes biodegradable waste to produce biogas rich in methane and carbon dioxide, and a digestate which may comprise an anorganic effluent and/or a stable solid residue.

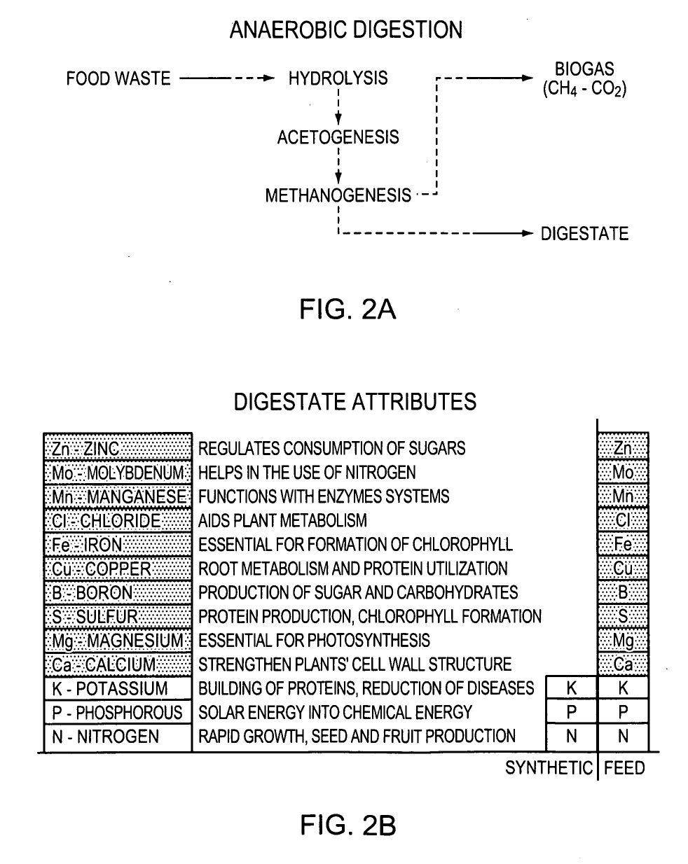

An exemplary anaerobic digestion process for use in a processing facility is shown in FIG. 2A. The digestion process begins with bacterial hydrolysis of the input materials, which breaks down insoluble organic polymers, such as carbohydrates and proteins, and makes them available for other bacteria. Acidogenic bacteria then convert the sugars and amino acids into carbon dioxide, hydrogen, ammonia, and organic acids, and acetogenic bacteria convert the resulting organic acids into acetic acid, along with additional ammonia, hydrogen, and carbon dioxide. Finally, methanogens are able to convert these products to methane and carbon dioxide. The remaining non-digestible material forms the digestate, which is typically rich in nutrients.

FIG. 2B shows a table listing digestate attributes for exemplary materials processed by a biodegradable waste-processing facility.

The success of waste treatment by anaerobic digestions depends on effective biomass retention. Standard reactor designs like continuously stirred reactors (CSTR) and plug-flow reactors (PFR) are susceptible to washout of the microbial mass, and are thus, absent any biomass retention apparatus, generally unsuitable for processing biodegradable waste. For certain wastewaters, biomass retention times can be increased through biomass granulation and/or biofilm formation. These biomass-retaining processes, however, typically work only for narrow ranges of hydraulic flow rates, and suitable concentrations of nutrients and suspended solids.

Anaerobic membrane bioreactors (anaerobic MBRs) provide alternative means for achieving nearly complete biomass retention, irrespective of the capacity of the biomass to form biofilms of granules. An anaerobic MBR utilizes one or more micro-filtration or ultra-filtration membranes to physically retain biomass inside the reactor, thereby eliminating the risk of biomass washout. More generally, the membrane acts as a filter passing solubilized components, but retaining solids of dimensions greater than a pore size of the membrane. As a result, solids-retention and hydraulic-retention times are decoupled, which increases waste-processing performance parameters (e.g., percentage of biodegradable material converted to biogas, percentage of COD destructed, methane fraction in biogas). Further, since an anaerobic MBR does not require as much energy for the regeneration of biomass, in contrast to bioreactors that incur substantial biomass losses, it can reach higher overall energy efficiency. In addition, an anaerobic MBR typically has a small footprint, compared with a conventional waste-processing bioreactors of the same processing capacity, because of its capacity to handle large organic loading rates.

Anaerobic MBR technology is suitable for high-strength wastewaters, i.e., wastewater with a high biological oxygen demand (BOD). Solid biodegradable waste streams, on the other hand, cannot, in general, be used readily as a feedstock for an anaerobic MBR because the solids would clog the membrane(s) and thus adversely affect the MBR performance. Further, solid waste streams are often contaminated by non-degradable or not easily degradable materials such as bone, shells, seeds, pits, glass, metal, plastic, etc. These contaminants can permanently damage the membrane(s), reducing the operational life span of the MBR. Further, contaminants in the digestate would preclude use of the digestate as fertilizer.

Various embodiments of the present invention enable the application of anaerobic membrane digestion to contaminated solid (in addition to liquid) waste streams by providing a pulper for the efficient separation of non-degradable contaminants from a mixed waste stream, and the solubilization of the biodegradable material. Mixed waste streams include, for example, food waste mixed with packaging material, municipal waste, industrial waste, and agricultural waste.

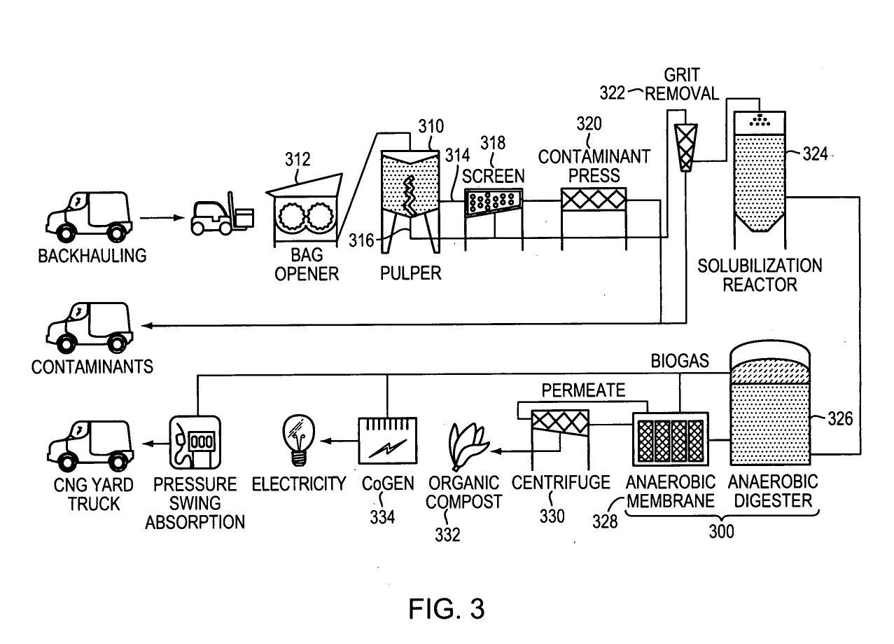

FIG. 3 depicts a waste-processing facility 110 including an anaerobic MBR 300 and a pulper 310, in accordance with various embodiments. The facility 110 may include a bag opener 312 that rips plastic and other packaging material, utilizing, e.g., weak shear forces, to release the biodegradable waste contained therein. The waste is then conveyed to the pulper 310, where the biodegradable material is disintegrated.

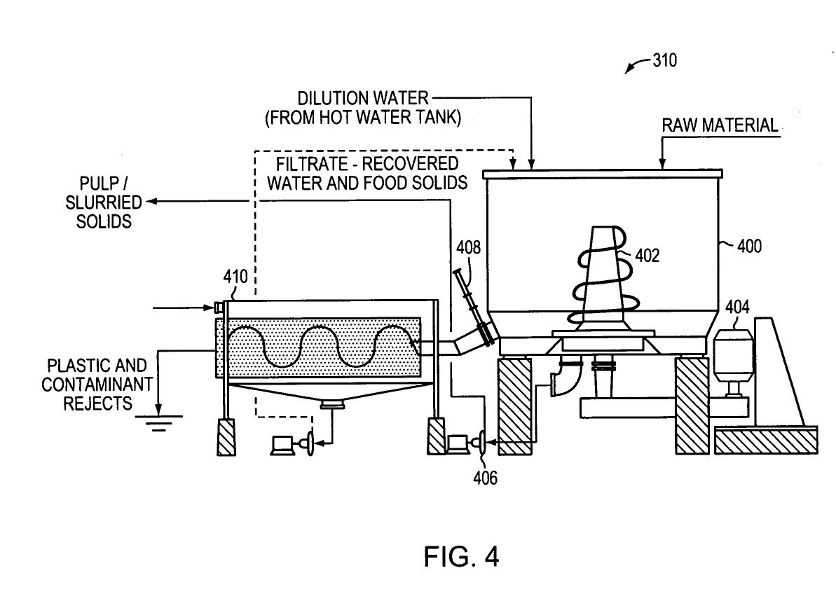

FIG. 4 illustrates an exemplary pulper 310 and contaminant removal system. The pulper 310 includes a tub-like tank 402, or stator, for holding the waste feedstock, and a rotor 404, e.g., a helical screw-type rotor, inside the stator for generating hydraulic shear to de-fiber the waste into a pulp. The rotor is driven by a motor 404. Pulping, as opposed to shredding, the waste largely preserves non-biodegradable waste components while solubilizing the biodegradable material. As a result, the non-biodegradable contaminants can readily be separated from the pulp suspension. In addition, preserving the structural integrity of contaminants such as, e.g., discarded batteries, avoids the release of toxins that could otherwise result from their destruction.

The pulper may process waste in batches according to the following operational sequence: After adequate amounts of hot water (e.g., of about 150° F.) and raw material have been added to the tank 402, the rotor 404 is turned on, and is run until the degradable material is largely broken down. Nearly full breakdown may be achieved in twenty minutes or less. Once the biodegradable material is sufficiently disintegrated, a discharge pump 406 is turned on to allow large fractions of the pulped material to leave the pulper through a perforated bedplate (not shown) located underneath the rotor 404. The bedplate perforations may be circular or elongated, and may vary in size depending on the type of waste processed. For food waste from retailers, a bedplate with 3/16-inch or ⅛-inch diameter perforations may be used. While the pulped material passes through the bedplate, most contaminants are held back due to their size. Once the majority of the pulp suspension has been removed, the tank may be refilled with hot water up to about a third of its height. Then, a side valve 408 is opened, and the remaining waste is discharged. Separation hardware 410 (e.g., a rotary sieve, a basket filter, a vibrating screen, a drum screen, or a rotating filter) is used to filter the contaminants, and recycle the water, including any residual pulped biodegradable material, to the pulper. Then, the side valve is closed, and the next batch of waste can be processed.

In some embodiments, a “light fraction” (e.g., plastics, bones, waxed cardboard, styrofoam) and a “heavy fraction” (e.g., metal, glass, batteries) of contaminants are moved separately from the pulper. The heavy fraction may be collected in a flushed trap located on the bottom of the pulper. Rotating scrapers may continuously scrape settled heavy material towards the trap. The trap may be configured with an upper and lower gate, wherein the lower gate includes the bedplate. When the lower gate is closed and the upper gate is open, heavy material is collected in the trap. By flushing the trap with process water, organic build-up may be prevented, and the collected materials cleaned. When the upper gate is closed and the lower gate opened, the heavy waste is discharged from the trap and conveyed to a disposal bin. The light fraction, which primarily contains mixed plastics, foams, and other buoyants, may be captured with a mechanically operated rake submerged into the pulp. Alternatively or additionally, the light waste may be discharged through a side valve, as described above.

With renewed reference to FIG. 3, the pulper 310 may separate a contaminant stream 314 from a suspension of pulped biodegradable waste 316. The contaminants 314 are filtered through a screen 318, compacted in press 320, and shipped off for recycling or disposal. The pulped biodegradable waste 316 is transferred to the anaerobic MBR 300. Since the suspension of pulped biodegradable material 316 may contain small pieces of sand and grit, which are non-biodegradable and could clog or damage the membrane of the MBR 300, the waste-processing facility may further include a grit removal apparatus 322 between the pulper and the anaerobic MBR 300. The grit removal apparatus 322 may, for example, be a hydrocyclone, which separates grit from the suspension by centrifugation. Sand and grit are diverted to the bottom of the hydrocyclone, where they fall into a screw conveyor, and are transferred into a container for disposal.

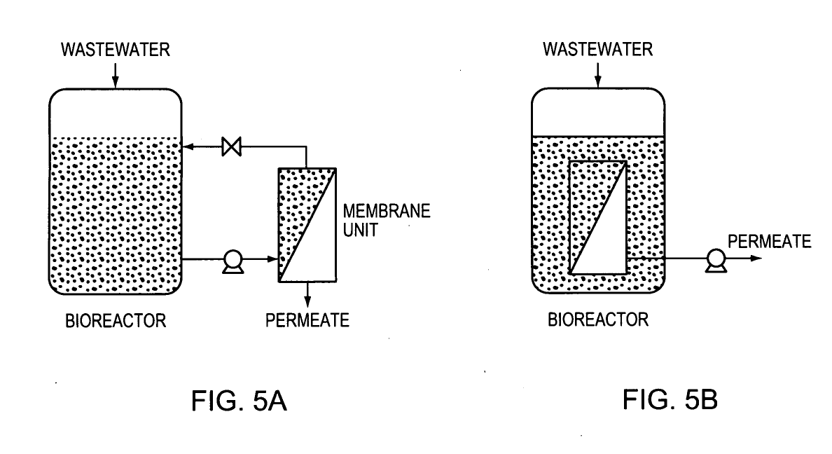

The grit-free suspension of pulp may be further solubilized in a solubilization reactor 324, or may instead be directly transferred to the anaerobic MBR 300, depending on the level of disintegration of the material achieved in the pulper. In the anaerobic MBR 300, microorganisms digest the biodegradable pulp into biogas and an anorganic effluent. The anaerobic MBR 300 includes a tank 326, and a membrane or set of membranes 328 (e.g., the KUBOTA submerged membrane unit developed by Kubota Corporation, Japan) submerged in the suspension. The membrane unit 328 performs biomass retention and gas/liquid/solids separation functions. In some embodiments, illustrated in FIGS. 3 and 5A, the membrane unit 328 is separate from the tank 326, requiring the suspension to be pumped through the membrane unit 328. In other embodiments, illustrated in FIG. 5B, the membrane unit 328 is located inside the tank 326.

The anaerobic digestion process results in two effluent streams: a sludge (typically representing about 10% of total effluent flow) and a permeate (typically representing about 90% of total effluent flow). The permeate has low suspended solid concentrations, but high concentrations of valuable nutrients and minerals. Therefore, it can be further refined to produce a valuable fertilizer as well as reclaimed process water, which may be recycled into the pulper. Recycling the hot water into the pulper bears the advantage that heat of the permeate is simultaneously transferred to the pulper. If the permeate contains high concentrations of nutrients, such as ammonia, these nutrients may be largely removed from the permeate prior to use as process water to avoid toxic levels. Ammonia are toxic to anaerobic digestion at about 3000 mg/l. An ammonia stripper may be employed to reduce ammonia concentrations to about 100 mg/l. Alternatively, the permeate may be used directly as liquid fertilizer and/or irrigation water. The sludge may be dewatered in a centrifuge 330, and composted to create a soil amendment 312; the extracted water may be added back into the MBR 300. The biogas generated in the MBR 300 may be converted to electricity and heat in a cogeneration engine 334.

In certain embodiments, the waste-processing facility is fully automated, requires little or no maintenance and user training, and enables a user to process waste and generate energy without changing current waste disposal behavior. Automated system management may be readily implemented using conventional equipment and techniques, and may involve the collection of performance data, such as internal pH, biogas production, and nutrient composition of fertilizer product, to assess the operation of the system and determine the necessary adjustments for optimization. For example, a pH-balancing unit may continually adjust the pH of the waste stream, thereby enabling diverse waste handling.

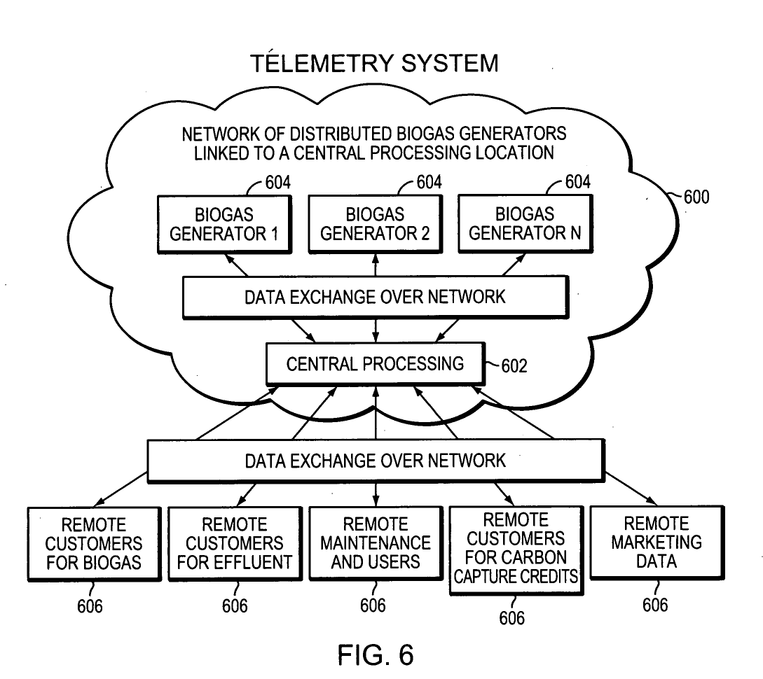

The automated management of the processing facility also facilitates its remote control. In some embodiments, the waste processing includes multiple integrated biogas generator units (each having at least an anaerobic MBR) for increased flexibility and reliability, and is an enclosed system remotely managed by a telemetry system, as illustrated for exemplary purposes in FIG. 6. The telemetry system 600 may include a central processing module 602 in communication with the biogas generator units 604. The central processing module 602 monitors the quality of byproducts and overall system performance, allowing it to quickly identify irregularities and diagnose malfunctions. The central processing module 602 may also give customers 606 access to critical data relevant to the performance of the system. Data that may be monitored includes, for example, daily and total waste amounts, waste by category, energy of input wastes, and waste disposal and processing trends. Managing the data remotely may result in a number of benefits for users, such as, but not limited to, waste savings, energy savings, public relations benefits, environmental, calculations, networked system management, improved operational performance, waste stream and efficiency analysis, and capacity management. For example, using the telemetry system 600, multiple processing facilities located at multiple distribution facilities may be monitored from a single remote location, allowing for efficient control and maintenance of a number of facilities with minimal input from the day-to-day users of the processing units.

Having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A system for processing waste comprising biodegradable material, the system comprising:
a pulper for removing non-biodegradable material from the waste and pulping the biodegradable material; and
an anaerobic membrane bioreactor comprising (i) microorganisms for producing biogas and anaerobic effluent from the pulped biodegradable material, and (ii) a membrane for retaining the microorganisms and a solid portion of the biodegradable material in the anaerobic membrane bioreactor,
wherein the removal of non-biodegradable material in the pulper protects the membrane of the anaerobic membrane bioreactor.

2. The system of claim 1, further comprising a hydrocyclone coupling the pulper to the anaerobic membrane bioreactor for removing grit from a suspension of the pulped biodegradable material prior to its introduction into the anaerobic membrane bioreactor.

3. The system of claim 1, wherein the anaerobic effluent comprises a sludge and a permeate comprising nutrients and minerals.

4. The system of claim 3, wherein water extracted from the permeate may be used as process water in the pulper.

5. The system of claim 1, further comprising a heater for heating an interior of the pulper so as to solubilize the biodegradable material.

6. The system of claim 1, further comprising waste containers for transporting the waste to the pulper, the waste containers comprising microorganisms for partially degrading the waste during transport.

7. The system of claim 1, wherein the waste comprises food waste.

8. The system of claim 1, wherein the waste comprises fat, oil, and grease.

9. A method of managing a waste stream comprising biodegradable material, the method comprising:
providing a waste-processing facility for processing biodegradable waste co-located with a distribution facility, wherein:
(a) the distribution facility receives food products from a producer and distributes the food products to at least one retailer; and
(b) the waste-processing facility comprises (i) a pulper for pulping biodegradable waste and (ii) an anaerobic bioreactor for producing biogas and anaerobic effluent from the pulped biodegradable waste; and
using the same vehicles both to deliver food products to a retailer from the distribution facility and to return biodegradable waste generated by the retailer to the waste-processing facility.

10. The method of claim 9, further comprising converting the biogas into at least one of fuel or electrical energy.

11. The method of claim 9, further comprising generating fertilizer from the anaerobic effluent.

12. The system of claim 9, further comprising using the same vehicles both to transport the food products from the producer to the distribution facility and to deliver fertilizer generated from the anaerobic effluent to the producer.

13. The method of claim 9, further comprising using energy generated from the biogas to heat the pulper.

14. The method of claim 9, further comprising monitoring the waste-processing facility from a remote location.

15. The method of claim 9, further comprising transporting the biodegradable waste from the retailer to the waste-processing facility in bioaugmented waste containers so as to partially degrade the biodegradable waste during transport.

16. The method of claim 9, wherein the anaerobic bioreactor is an anaerobic membrane bioreactor.

17. A method for processing waste, the method comprising:
removing non-biodegradable material from the waste and pulping the biodegradable material; and
anaerobically processing the pulped biodegradable material by (i) exposing it to microorganisms for producing biogas and anaerobic effluent from the pulped biodegradable material, and (ii) retaining the microorganisms.

18. The method of claim 17 wherein the anaerobic processing step takes place in an anaerobic membrane bioreactor.

19. The method of claim 18 wherein the removal of non-biodegradable material protects the membrane of the anaerobic membrane bioreactor.
